(19) United States
(12) Patent Application Publication
(10) Pub. No.: US 2004/0111219 A1
(43) Pub. Date: Jun. 10, 2004

(54) Active interferometric signal analysis in software

(76) Inventor: Sandeep Gulati, La Canada Flintridge, CA (US)

Correspondence Address: CARL A. KUKKONEN, III, C/O WUESTHOFF & WUESTHOFF, SCHWEIGERSTRASSE 2, MUNICH, 81541, DE

(21) Appl. No.: 10/616,869

(22) Filed: Jul. 9, 2003

Related U.S. Application Data
(63) Continuation-in-part of application No. 09/913,132, filed on Aug. 9, 2001, now Pat. No. 6,671,625.
Application No. 09/913,132 is a 371 of international application No. PCT/US00/04076, filed on Feb. 17, 2000.
Application No. 09/913,132 is a continuation of application No. 09/253,789, filed on Feb. 22, 1999, now Pat. No. 6,136,541.

(51) Int. Cl. G06F 19/00; G01N 33/48; G01N 33/50; G01N 31/00

(52) U.S. Cl. 702/19; 702/22; 702/20

(57) ABSTRACT

Techniques are provided for performing active interferometric signal analysis in software. The techniques exploit expressor functions designed to extract spectral invariants of events of interest associated with an arrayed platform device used to detect the signal pattern to be analyzed. Various techniques for generating expressor functions are also provided. Depending upon the implementation, the techniques provide for both detection and quantitation analysis by exploiting either constructive or destructive interferometric analysis using reverberant convergence to detect resonance events. The techniques achieve software emulation of wave-particle interactions and wave-wave interactions and can operate in either the frequency domain or the phase domain. The techniques may be used for analyzing static spatial systems, static data from arrayed measurement platforms, dynamical systems, spatio-temporal systems or plasma systems.

### RELATED APPLICATIONS

This application is a continuation-in-part of U.S. patent application Ser. No. 09/913,132, filed Aug. 9, 2001, entitled “Method and System for Signal Detection in Arrayed Instrumentation Based on Quantum Resonance”, which is a national stage application of PCT Application No. US00/04076 filed on Feb. 17, 2000, which claims priority from U.S. patent application Ser. No. 09/253,789, now U.S. Pat. No. 6,136,541, filed on Feb. 22, 1999, entitled “Method and Apparatus for, Analyzing Hybridized Biochip Patterns using Resonance Interactions Employing Quantum Expressor Functions, all of which are hereby fully incorporated by reference”. Also incorporated herein by reference in their entirety are the following patents based on applications filed contemporaneously with U.S. patent application Ser. No. 09/253,789: U.S. Pat. No. 6,142,681, entitled: “Method and Apparatus for Interpreting Hybridized Bioelectronic DNA Microarray Patterns Using Self-scaling Convergent Reverberant Dynamics” and U.S. Pat. No. 6,245,511, entitled “Method and Apparatus for Exponentially Convergent Therapy Effectiveness Monitoring Using DNA Microarray Based Viral Load Measurements”. Also incorporated herein by reference in their entirety are the following provisional patent applications: U.S. patent application Ser. No. 60/395,074, filed on Jul. 9, 2002, entitled “Active Signal Processing Technique Using Resonance Interferometry”. U.S. patent application Ser. No. 60/399,227, filed on Jul. 29, 2002, entitled “High-Throughput Quantitation of Signals from Arrayed Platforms Using Resonance Interferometry” U.S. patent application Ser. No. 60/408,057, filed on Sep. 3, 2002, entitled “Technique for Analysis and Quantitation of Arrayed Information Structures”. U.S. patent application Ser. No. 60/408,844, filed on Sep. 5, 2002, entitled “System and Method for Analysis and Quantitation of Arrayed Platforms”. U.S. patent application Ser. No. 60,440,253, filed on Jan. 15, 2003, entitled “Technique for Detection and Quantitation of Arrayed Platform Signals”.

### FIELD OF THE INVENTION

The invention generally relates to active signal analysis systems, and in particular, interferometric signal analysis systems.

### BACKGROUND OF THE INVENTION

1. Overview of Passive and Active Interferometric Signal Analysis

Interferometric analysis has proven useful for performing signal analysis, particularly for detecting events of interest within signal patterns, such as within a two-dimensional image. One example of interferometric analysis involves irradiating a moving object, such as an aircraft, with radar pulses, that interact with the object and interfere with one another and which are then reflected back to a sensing device such as an antenna. Signals received by the antenna are compared against signals originally transmitted in an effort to detect a unique spectral signature of the object so as to identify the object. This form of interferometric analysis is referred to as “active analysis”, because signals used to irradiate the event of interest interact with the object and with other signals to actively modify the resulting interference pattern. In other words, the system being analyzed is actively modified. In the case of radar, electromagnetic fields surrounding the event of interest are actively modified.

An alternative type of signal analysis is “passive analysis” wherein signals that have already been detected are processed in an effort to identify events of interest therein. Examples include filtering images in an attempt to enhance features therein. These techniques are deemed to be passive because no noise or other signals are added into the system being analyzed. More specifically, passive interferometric analysis typically involves the manipulation of observed intensity patterns or phase information without the addition of noise or other signals. Often, passive analysis systems exploit parametric methods such as matched filter techniques, maximum a-posteriori (MAP) techniques, maximum likelihood estimator (MLE) techniques, singular value decomposition (SVD), classical Fourier analysis, parametric distributional clustering (PCA), or median filter variants. Non-parametric methods may be employed as well. In addition, quantitation and clustering techniques may be employed such as “soft-computing” and its variants which include neural networks, fuzzy logic systems, and Bayesian inferencing systems. Although a wider range of passive analysis techniques have been exploited, each requires that the observed signal have an intensity greater than the intensity of the background signal in which it appears. In addition, the analysis system must properly estimate the background signal and to properly estimate any variability in the background signal. In some cases, spread spectrum (S-S) amplitude correlation is provided with background subtraction, and scaling and normalization is employed to compensate for detector nonlinearities. Hence, there are various limitations to passive analysis, particularly the inability to detect signals having an intensity less than or near to the intensity level of the background.

Insofar as the analysis of hybridized biological microarray patterns is concerned, heretofore, passive analysis has been the only form of analysis performed. Briefly, a signal pattern output from a biological microarray is normalized to compensate for dye, scanner, or array effects. Then an estimate is made as to the background signals at feature, transcript or chip levels. The background is then subtracted from the foreground and a ratio of foreground minus background is calculated to quantitate a differential expression representative of patterns of interest. Often, this calculation is performed using channel ratios. In any case, examples of passive analysis of hybridized digitized image patterns include PCA, SVD, MLE, hidden Markov Model (HMM), and various statistical approaches. As the technique is passive, it is subject to the limitations summarized above, in particular, only hybridized transcripts with genes above the estimated background can be detected. In addition, high call variability in targets with low expression levels arises and high CV in fold change computations occurs. Skewed results can arise when dealing with platforms or protocols having a limited dynamic range. Typically, passive hybridization analysis techniques require vast amounts of data and exemplars to allow refinement of statistics driving the call analysis. Heretofore, efforts to improve the accuracy and reliability of passive techniques for analyzing hybridized digitized images involve wet signal amplification and biological enhancements. Computational enhancements of the passive analysis have been fairly minimal.

Thus, heretofore, computer systems have been largely limited to performing passive analysis whereas active analysis is largely performed only by hardware systems.

2. Hardware-Based Active Signal Analysis

With hardware-based active interferometric signal analysis, external excitation is provided to the system being analyzed while the system is being analyzed to provide an interaction that generates additional information or provides some change in a qualitative property of the system. Typically, the external excitation involves some form of noise injection, which results in a nonlinear coupling between the noise and events of interest within the system being analyzed, which enhances those events. As noted, one example of active signal processing (ASP) is radar wherein the signal being injected into the system being analyzed is the - radar signal. Another example is the superconducting quantum interferometric device (SQUID), which utilizes the quantum effects of superconductivity to detect magnetic fields. Other examples include imagery intelligence (IMINT), signals intelligence (SIGINT), electronic intelligence (ELINT) devices. In addition, active signal processing has been applied to femto-lasers. While these hardware-based systems provide enhanced signals in some circumstances, such systems are expensive to build and operate and can only physically manipulate or generate very specific types of signals.

3. Stochastic Resonance Techniques

Another exemplary active system or technique that has been implemented in software is stochastic resonance wherein noise is applied to an image or a system being analyzed to improve at output signal-to-noise ratio (SNR). With stochastic resonance, a detector is modeled in terms of a bistable nonlinear dynamic system, which is enhanced by applying additive random noise element and a periodic sinusoidal forcing function. Stochastic resonance occurs when the SNR passes through a maximum as the noise level is increased. By properly selecting the amount of noise being applied, events of interest can be enhanced so that signals below background can be detected.

Traditionally, stochastic resonance is employed using a classical noise source such as a standard Gaussian noise pattern. Alternatively, however, stochastic resonance may employed using some form of quantum noise. In this regard, it has been shown that irradiation of a bistable system with a weak resonance laser field induces long-lived coherent tunneling motion whose amplitude exhibits a quantum stochastic resonance (QSR) as a function of the two-level-system relaxation rate. and the field strength (See Makarov D E. Makri N. “Stochastic resonance and nonlinear response in double-quantum-well structures” Physical Review B. 52(4):R2257-R2260, 1995 Jul. 15). Essentially, QSR is an enhancement of the response of a driven quantum mechanical system by quantum noise.

In any case, heretofore, traditional stochastic resonance and QSR have only been applied to point dynamical systems, i.e. to systems providing only a signal data point varying in time. One other technique for enhancing stochastic resonance is so-called array-enhanced stochastic resonance (AESR), which extends stochastic resonance to 2-D dynamical systems, though still limited to static systems. Although an improvement, AESR is typically limited to the analysis of systems with relatively few degrees of freedom.

Thus, most conventional active signal analysis systems are not implemented in software and those that have been implemented in software typically have significant limitations. Accordingly, it would be desirable provide new or improved techniques for implementing active signal processing in software and it is to these ends that aspects of invention are drawn. By implementing active signal analysis in software, all of the advantages associated with software systems over hardware systems can be exploited, including lower cost, increased flexibility, increased robustness, portability, etc.

More specifically, it is desirable to provide an active signal processing system implemented in software capable of detecting events of interest in spatial, temporal or spatio-temporal systems within a combinatorial state space wherein the state space is discrete and continuous and wherein background noise is nonstationary. Ideally, the system would be capable of analyzing signals received from a wide variety of arrayed platform detector configurations including linear, arrayed, spectral, and time-varying and can handle N-attribute systems. Moreover, ideally, the improved system would be capable of analyzing signals with a signal to background ratio of 10⁻⁴. Insofar as the analysis of biological samples applied to a microarray platform is concerned, the system or technique would ideally be capable of detecting and quantitating specific hybridization (e.g., genomic events) and specific binding events (e.g., proteomic events). Insofar as genomics is concerned, the system should be capable of processing data from a high-density gene expression microarrays (i.e. array is providing 10² to 10⁶ spots per array) and which represents discrete, spatially indexed systems having high probe densities (i.e. from 10⁶ to 10⁹ probes per spot).

### SUMMARY OF THE INVENTION

In accordance with the invention, techniques are provided for performing active interferometric signal analysis in software. Depending upon the implementation, the techniques provide for both detection and quantitation analysis by exploiting constructive or destructive interferometric analysis (or a combination of constructive and destructive interferometric analysis) using reverberant convergence to detect resonance events. The techniques achieve software emulation of wave-particle interactions and wave-wave interactions and can operate in either the frequency domain or the phase domain. The techniques may be used for analyzing static spatial systems, static data from arrayed measurement platforms, dynamical systems, spatio-temporal systems or plasma systems.

Briefly, the techniques exploit expresser functions designed to reject any interfering noise or background clutter from possible events of interest within arrayed data to be analyzed. More specifically, the expresser functions are designed so as to extract spectral invariants of possible events of interest associated with the array device used to detect the arrayed data. Various aspects of the invention are directed to the generation of the expressor functions. Typically, the expressor functions are designed to incorporate some form of non-Gaussian noise, such as quantum noise, pseudorandom noise, polycyclostationary noise, cyclostationary noise, stationary noise, non-stationary noise or a systemic bias. Preferably, prior to the application of the expresser function to arrayed data, the array is pre-conditioned so as to convert the arrayed data to a spectral domain in which spectral harmonics parameterize events of interest to a pre-determined dynamical system. Preconditioning may be performed by applying a preconditioning function incorporating, e.g., a 1-D Fourier function, a 2-D Fourier function, an N-D Fourier function, a time division multiplexing (TDM) function, a wavelength division multiplexing (WDM) function, a frequency division multiplexing (FDM) function, a radial bias function, a wavelet kernel function, a fractal function, or a soliton function. The arrayed data itself may be the form of a spatial 1-D array, a spatial 2-D array, a spatial point emitter array, a temporal point emitter array, a spatio-temporal point emitter array, a spectral point emitted array or a virtual array constructed by combining spatial separate point emitters.

In accordance with an exemplary method, a technique is provided for actively analyzing a signal pattern representative of arrayed data to identify events of interest therein. A signal pattern representative of arrayed data is input and resonance patterns are generated based on interference between synthetic noise and the signal pattern. Next, resonances within the resonance patterns associated with events of interest are detected. Preferably, the signal pattern is preconditioned prior to the step of applying synthetic noise to the signal pattern.

In a first exemplary system embodiment, also referred to herein as an “open loop interferometric system”, a system is provided for analyzing an arrayed signal pattern generated by an arrayed platform device to identify events of interest within the signal pattern. The system includes an expressor function input unit for inputting previously generated expresser functions capable of extracting spatial, spatio-temporal or spectral invariants of events of interest associated with the particular arrayed platform device being used. The system also includes a preconditioning unit for preconditioning the arrayed signal pattern. An active interferometric coupler operates to convolve the preconditioned arrayed signal pattern and the expresser functions so as to interferometrically enhance portions of the preconditioned pattern associated with events of interest, if any, present within preconditioned pattern. The system also includes a resonance marker detector for identifying the occurrence of events of interest within the convolved signal pattern.

In a second exemplary system embodiment, also referred to herein as a “goal-directed interferometric system”, the system for analyzing an arrayed signal pattern includes an expressor function input unit for inputting previously-generated expressor functions and an adaptive interferometric coupler for preconditioning the arrayed signal pattern so as to convert the arrayed signal pattern to a spectral domain while simultaneously convolving the signal pattern and the expressor functions so as to interferometrically enhance portions of the signal pattern to identify events of interest, if any, within the signal pattern.

In a third exemplary system embodiment, also referred to herein as a “self-organizing interferometric system”, the system includes a preconditioner for preconditioning the arrayed signal pattern and an expresser function adaptation unit for generating preconditioned expresser functions based on input canonical expresser functions (i.e. general expressor function not associated with the particular platform being used) and based on a preconditioned signal pattern while interferometrically enhancing portions the preconditioned signal pattern so as to identify events of interest, if any, within the signal pattern.

In a fourth exemplary system embodiment, also referred to herein as an “iterative interferometric system”, the system includes an expresser function input unit for inputting previously-generated expresser functions derived for the platform being used, a preconditioner for preconditioning the arrayed signal pattern and a iterative convolution coupler. The system also includes an adaptive controller unit for controlling the coupler to iteratively and selectively convolve the expresser functions to a preconditioned signal pattern until a predetermined degree of convergence is achieved so as to identify events of interest within the enhanced signal pattern.

A variety of techniques are set forth for performing active signal analysis in software. Method, system and computer product embodiments are provided.

### BRIEF DESCRIPTION OF THE DRAWINGS

The features, objects, and advantages of the present invention will become more apparent from the detailed description set forth below when taken in conjunction with the drawings in which like reference characters identify correspondingly throughout and wherein:

FIG. 1 is a block diagram providing an overview of system components of the first exemplary embodiment of the invention (the open loop interferometric system), which provides separate expresser function generation unit, preconditioner, active interferometric coupler and resonant marker detector components;

FIG. 2 is a flowchart providing an overview of method steps for the first exemplary embodiment of the invention;

FIG. 3 is a block diagram providing an overview of system components of the second exemplary embodiment of the invention (the goal-directed interferometric system), which provides an adaptive interferometric coupler;

FIG. 4 is a flowchart providing an overview of method steps for the second exemplary embodiment of the invention;

FIG. 5 is a block diagram providing an overview of system components of the third exemplary embodiment of the invention (the self-organizing interferometric system), which provides a expressor function adaptation unit;

FIG. 6 is a flowchart providing an overview of method steps for the third exemplary embodiment of the invention;

FIG. 7 is a block diagram providing an overview of system components of the fourth exemplary embodiment of the invention (the iterative interferometric system), which provides a adaptive controller for iteratively controlling a separate iterative interferometric coupler;

FIG. 8 is a flowchart providing an overview of method steps for the fourth exemplary embodiment of the invention;

FIG. 9 is a graph illustrating combinations of arrayed data, signal patterns that may be processed by any of the embodiments of the invention; and

FIG. 10 is a graph illustrating combinations of preconditioning functions and expresser functions that may be used in connection with any of the embodiments of the invention;

FIG. 11 is a graph illustrating an exemplary platform array detector and active interferometric analysis system that may exploit the invention;

FIG. 12 is a graph illustrating various platform array detectors that may be used in connection with any of the embodiments of the invention;

FIG. 13 is a graph illustrating the structure of an exemplary microarray platform that may be used to generate arrayed data for use by any of the embodiments of the invention; and

FIG. 14 is a flowchart summarizing the overall method of the invention, at least in accordance with an exemplary embodiment thereof.

### DETAILED DESCRIPTION OF EXEMPLARY EMBODIMENTS

With reference to the remaining figures, exemplary embodiments of the invention will now be described.

A. Open Loop Interferometric System

1. Overview

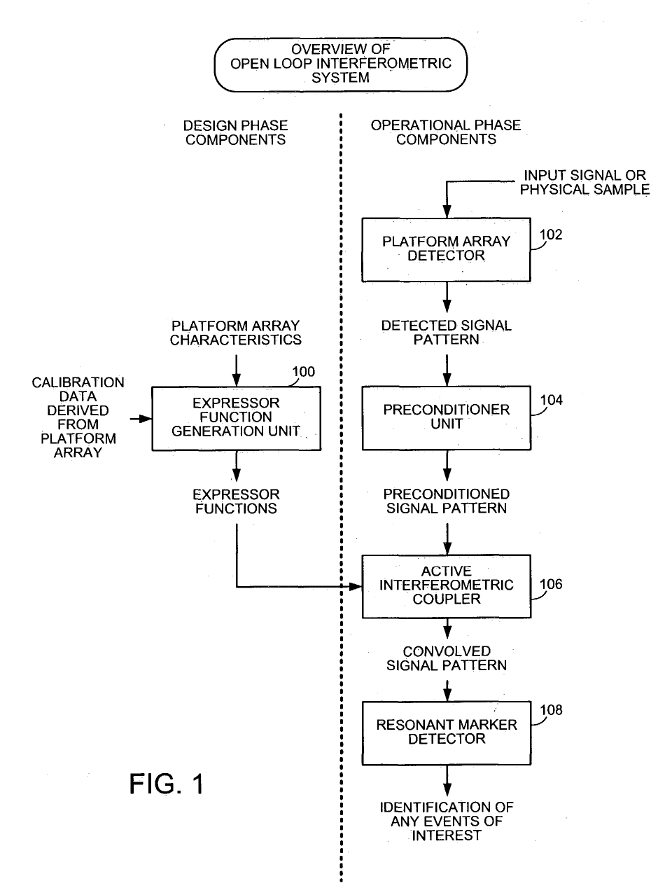

FIG. 1 illustrates, at a high-level, exemplary system components for the first exemplary implementation of the invention. Components employed only during a design phase are shown on the left; components employed during the actual analysis of an input signal are shown on the right. Briefly, during the design phase, expresser function generation unit 100 generates one or more designer expressor functions based upon the specific characteristics of a platform array detector 102 to be used to detect signal patterns to be processed and also based on calibration data derived from platform array 102. The calibration data is generated based on known events of interest. That is, input signals or physical samples containing known events of interest are applied to the detector to generate the calibration data. In one example, the platform array detector is a genomic biochip/microarray and the calibration data is derived from a biological sample containing known gene expressions. The characteristics of the platform array detector that are input by the expressor function generation unit include both the layout of the array as well as its coherent noise or background signal characteristics. Once the expressor functions have been generated for the particular platform array detector based on the calibration data, the design phase is complete and, thereafter, the expresser function generation unit is no longer required unless an alternative platform array detector is employed.

Components employed during the actual operational phase of the system include the aforementioned platform array detector 102 that generates a detected signal pattern and a preconditioner unit 104 that preconditions the detected signal pattern so as to convert the signal pattern to a spectral domain (for an example of a technique for transforming the signal pattern to a spectral domain, please see U.S. patent application Ser. No. 10/430,664 entitled “Method and System for Characterizing Microarray Output Data” filed on May 5, 2003 which is hereby fully incorporated by reference) in which spectral harmonics parameterize events of interest to a predetermined dynamical system (for which the applicable characteristics are input into the preconditioner unit. The operational phase components also include an active interferometric coupler 106 that convolves the preconditioned signal pattern and the previously generated expresser functions. The convolution is performed so as to interferometrically enhance portions of the preconditioned signal pattern associated with events of interest, if any, that are present within the preconditioned signal pattern. As explained more fully below, the convolution is performed via reverberant convergence so as to emulate active interferometric enhancement. Finally, the system also includes a resonant marker detector 108 that processes the convolved signal pattern so as to identify particular events of interest, if any, appearing within the convolved signal pattern.

With the exception of the platform array detector, which is a physical hardware component, all other components illustrated in FIG. 1 may be implemented in software, hardware, firmware or some combination thereof. In particular, each component may be a computer product operative to perform the functions described. In one example, each component is a software module operating within a single generally programmable computer. In other cases, the software modules are implemented using separate microprocessors or application specific integrated chips (ASICs). In still other examples, some of the components are implanted in software whereas others are completely hardwired. As can be appreciated, a wide range of possible implementations is consistent with the invention and no attempt is made herein to describe all possible configurations.

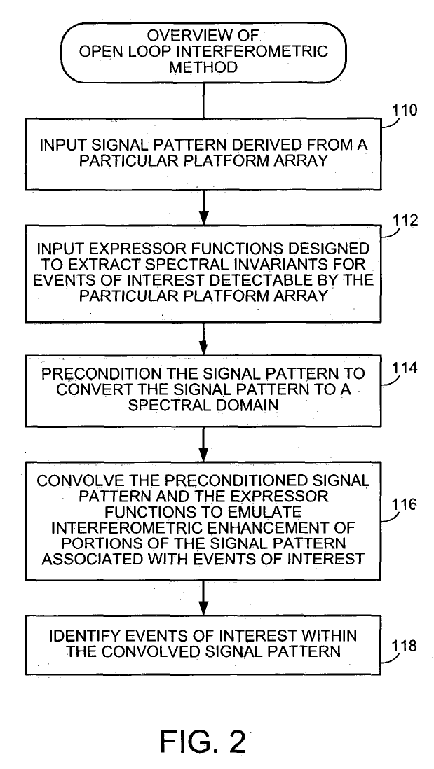

Method steps associated with the open loop interferometric system of FIG. 1 will now briefly be summarized with reference to FIG. 2. Although these steps are advantageously performed by the system of FIG. 1, the steps can be performed by any other suitable system. Conversely, the system of FIG. 1, or portions thereof, may be employed to perform methods different from that which is illustrated in FIG. 2. The same is true for the various other figures of the patent application. In any case, at step 110, a signal pattern derived from a particular platform array detector is input and, at step 112, expresser functions designed to extract spectral invariants for events of interest detectable by the particular platform array are input. Then, at step 114, the input signal pattern is preconditioned to convert the signal pattern to the spectral domain in which spectral harmonics parameterize events of interest to the aforementioned predetermined dynamical system. At step 116, the preconditioned signal pattern and the expresser functions are then convolved using reverberant convergence so as to emulate an active interferometric enhancement of portions of the signal pattern associated with events of interest thus yielding a convolved signal pattern (also referred to herein as an enhanced signal pattern. Finally, at step 118, the convolved signal pattern is examined to identify events of interest therein.

It should be noted that a list of specific events of interest need not be input at step 112. Rather, the technique operates to detect possible events of interest present in the input pattern based solely on the input pattern and the previously-generated expresser functions. Hence, the technique of FIG. 2 is not merely a diagnostic technique that seeks to determine whether particular pre-determined events of interest are present in the input signal pattern. This will be more readily apparent from the detailed examples which follow.

2. Exemplary Implementation

The mathematical equations performed by the various components of FIG. 1 or during the various method steps of FIG. 2 will now the set forth in detail. In this example, quantum expressor functions are employed. However, as described below, other expresser functions can alternatively be used, with the equations modified as needed dependent upon factors such as signal to background strength, extraction core length of the events of interest being analyzed, platform coherence, and desired sensitivity.

The exemplary equations are particularly relevant for conditions where the background is 10 to 1000 times stronger than the signal.

Initially, during the design phase, expresser function generation unit 100 of FIG. 1 inputs platform array detector characteristics represented mathematically as:

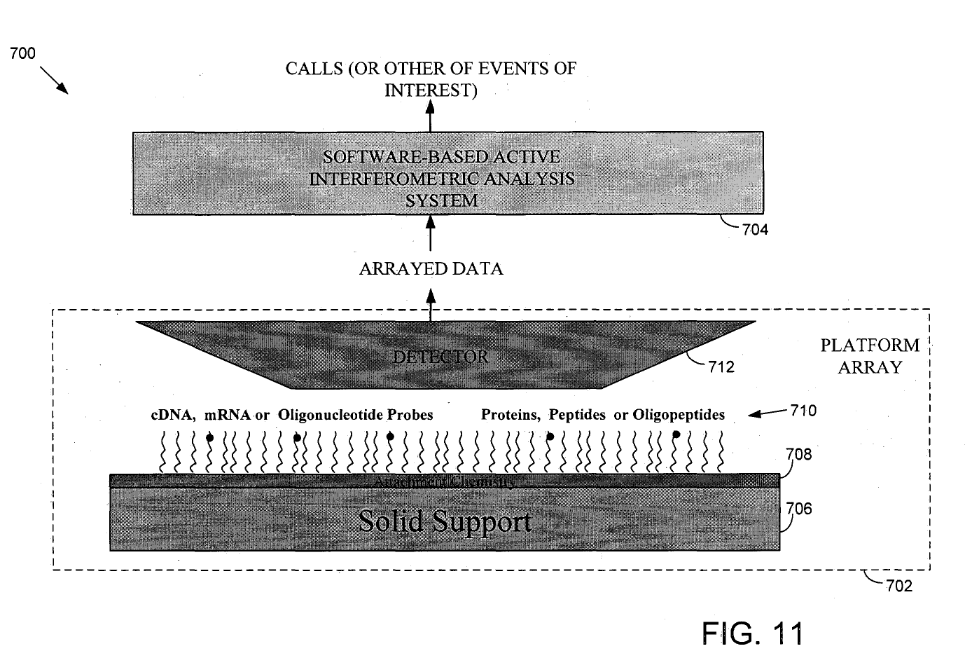

Ψ[Ω(N,M,T,Λ)]

where Ω(N,M,T,Λ) denotes a preconditioned extraction core corresponding to one or more events of interest. The indices N (0:n), M(0:m), T(1:k), A(0:p) respectively refer to the spatial, temporal and spectral dimensions of the physical or virtual arrayed platform described in FIG. 11. Details of preconditioning the extraction core corresponding to event of interest are given in U.S. patent application Ser. No. 10/430,664 entitled “Method and System for Characterizing Microarray Output Data” filed on May 5, 2003.

The Expressor Function Generation unit also inputs calibrated data (j exemplars) represented mathematically as:

$${⋃}_{j} {Ψ}_{1} {Ψ}_{2} {Ψ}_{3} … {Ψ}_{j} .$$

Based on the input calibrated data, the expressor function generation unit performs calculations provided below.

In some embodiments, Quantum Expressor Functions (QEF's) are calculated based upon the signal pattern based upon one or more events of interest. The QEF is generated by the steps of:

(a) selecting a canonical coupled Spin Boson basis system by first conditioning a Hamiltonian for that system;

(b) calculating harmonic amplitudes |Pₘ| for the Hamiltonian

(c) generating an order function (OF);

(d) measuring the entrainment states of the OF using exemplars with ground truth data; and

(e) modulating the OF using calibrated exemplars to yield the QEF

Fully characterized exemplars of the event of interest are used to develop a platform specific QEF. Hamiltonians are utilized as they provide a mathematical basis for determining the dynamical behavior of a system at each coordinate and at each momentum. Hamiltonian systems are further characterized by kinetic and potential energy. A key property of Hamiltonian systems is that they are conservative and have no dissipation of energy during relaxation. The use of a Spin Boson Hamiltonian, which is a particular type of Hamiltonian systems, permits the exploitation of quantum stochastic resonance phenomenonology. Hamilton's Equations and Hamiltonian functions pertain to well understood concepts in dynamical systems theory and dynamical equations of motion. (See “Theory and Problems of Lagrangian Dynamics”, Ed. Dare A. Wells, Schaum's Outline Series, McGraw-Hill Company, NY, 1967, pages 316-321; Elements of Hamiltonian Mechanics”, Dter Haar, Pergamnon Press, 1960, pages 95-102; and “Statistical Mechanics”, R. K. Pathria, Pergamon Press, Toronto, 1972, pages 6-7, 136-147).

The QEF encodes quantum noise to drive order in an asymmetric bistable quantum-mechanical system. The appearance of a resonance requires an asymmetry in the energies of the two states. A rate equation constructed for transitioning between the two states of a bistable system, such that the dynamics can be characterized in terms of transition rates Φ+ and Φ− between two asymmetric quantum superposition states where the drive frequency and interwell transition rates are much slower than intrawell relaxation rates. The signal to noise ratio (SNR) of such a superposition system is given by:

$$SNR = \frac{π}{4} {\frac{{Φ}_{+ 0}}{1 + exp [ {ϵ}_{0} / {κ}_{B} {T}_{0} ]} [ δ [ \frac{ϵ}{{κ}_{B} T} ] ]}^{2} ϵ$$

Where κ_{B} is the Boltzmann's constant, T is the temperature, and the sinusoidally modulated asymmetry energy ε is given by:

ε=ε₀+δ_{ε}×cos ωₛt

C(τ)=<n₊(t+τ\q₊,t)n₊(t\q₀,−∞)>

For the above bistable system, the power spectrum S(ω) may be represented by the Fourier transform of C(τ), containing a roughly Lorentzian broadband noise background and δ-function peaks at ω=0, the driving frequency ωₛ, and its harmonics. A measured correlation function for the quantum noise is given by C(t)=<n₊ᵢ(t)n₊₁(t⁺ τ)>, where each n₊₁=0 or 1, and the <, indicates an average over t over many data points i taken at equal intervals, and is given by

The probability of being in the + quantum state at t after being in the state q₀ is given by

n₊(t\q₀,−∞).

A custom Hamiltonian, couples the above system to an ensemble of harmonic oscillators is given by:

$$H = \frac{1}{2} {ϵσ}_{Z} - \frac{1}{2} {ℏΔσ}_{x} + {σ}_{y} {\sum }_{η} {V}_{μ} ( {ξ}_{η} + {ξ}_{η}^{ℏ} ) + ℏ {\sum }_{η} {ω}_{η}$$

In the above Hamiltonian, ε denotes the asymmetric energy, Δ is the tunneling matrix element, and σᵢ are Pauli spin matrices, and ζ_{η} is a harmonic oscillator creation operator with frequencies ω_{η}.

An important aspect is to couple the transformed and preconditioned discrete microarray output to a mathematical model for a quantum-mechanical dynamical system with specific properties. Specific exemplary parameters for use in calculating the Hamiltonian are those proposed by A. J. Legett et al., Reviews of Modern Physics, 59, 1, 1987 and A. O. Caldiera and A. J. Legett Annals of Physics, 149, 374, 1983. The parameters are important for an offline simulation of this Spin Boson system on a digital computer. The empirical observables are then collected and used to estimate and compute spectral properties, which are actually used by the method.

Experimental or analytical valid parameters for the above system will work with the technique because the robustness of the method depends only on the bulk and qualitative properties shown by this system and the properties of its power spectral harmonics. This is an important because the method is substantially immune to nuances and specifies of the actual driving mathematical system.

The information about the effects of the environment is contained in the spectral density J(ω)

J(ω)=(π/2)×Σ_{η}V_{η}²δ(ω−ω_{η}).

The harmonic amplitudes determine the weights of δ spikes of an averaged spectral power density in an asymptotic state S^{∞}(ω). ε refers to the coupling strength between the spin and boson systems, and P₀ defines the equilibrium state in the absence of driving force. Power Spectral Density (pSD) is a well understood concept is signal processing and basis for many computational algorithms. For a spectral signature, partitioned into bands of interest, power spectral density is formally as the set of measurements of average power in each spectral band, normalized by bandwidths. Formal definition can be found in “Cyclostationarity in Communications and Signal Processing”, Ed. William Gardner, 1994, IEEE Press, NY, pages 46-47.

The power amplitudes ηₘ in the mth frequency component of asymptotic state space are calculated using

ηₘ(Ω, ε)=4π|Pₘ(Ω, ε)|

and the phase shift is given by

$${ϕ}_{m} ( Ω , ϵ ) = {tan}^{- 1} [ \frac{Im ( {P}_{in} ( Ω , ϵ ) )}{{Re P}_{m} ( Ω , ϵ )} ] .$$

The analytic for the external force is given by

$${P}_{m} ( Ω , ϵ ) = \frac{γ}{γ - im Ω} \frac{2 {ω}_{c}}{π} h ( - im Ω , γ )$$

The parameters γ, ε₀ are predetermined and are design specific. Typically, values of 0.001 and 0.0001 are used for γ and ε₀ respectively. In the above expression |Pₘ| determine the weights of the δ spikes of the averaged spectral power density.

For some embodiments, the QEF is designed by matching the power spectral density (PSD) amplitude of preconditioned, extracted events of interest to that of the Spin Boson system described above so that stochastic and deterministic time scales match and so that the time scales couple back to noise statistics and degree of asymmetry. The technique employs a fully automated iterative conjugate gradient relaxation method for spectral matching between asymmetric base system and the transformed, precondiioned extraction core. The determination of the QEF depends on the specifics of bioelectronics substrates used for actual analysis. The method is however generalizable to all arrayed embodiments. In addition, the method is highly scalable to array dimensions (as the offline design trade-space time does not matter to computational complexity). As the system is an overdetermined coupled system, convergence criteria and stability of relaxation method directly relates to downstream resonance effectiveness.

Generation of Order Function

The order function (OF) of ground truth is generated as follows. The order function (OF) is for ground truth wherein ground truth represents a state wherein a positive signal to noise ratio (SNR) is expected for pixel intensities of selected exemplars used for developing an QEF.

A classical Order Function (OF) is used with the following form:

$$H ( θ ) = - {\sum }_{k = - ∞}^{∞} {h}_{k} {Z}_{k} {ⅇ}^{- 2 π ikθ}$$

$$where i = \sqrt{- 1} and {h}_{k} ≡ {\int }_{0}^{1} ⅆ θ h ( θ ) {ⅇ}^{- 2 π ikθ} and 0 < θ < 1.$$

The term

$${h}_{k} ≡ {\int }_{0}^{1} ⅆ θ h ( θ ) {ⅇ}^{- 2 π ikθ}$$

is referred to as the Diado integral.

In this application, QEF are designed from a canonical dynamical system drawn from a family of exponential systems. Daido integral is a specific term used to construct an Order Function, which is then used to construct the QEF.

The OF is calculated using an order match which implies that the variance of the density function for a specific exponential family approaches 0 (within 0.000001-0.0001). The free energy for the density function is given by

$$p ( x ) = \frac{1}{\sqrt{2 π}} {\int }_{k = - ∞}^{∞} {ξ}^{a} {ξ}_{b} {ⅇ}^{[ iλ ( {X}_{a}^{b} - x {δ}_{a}^{b} ) ]} ⅆ λ$$

where ζ^{a} and ζ_{b} represent state vectors. Xₐ^{b} represents the random observable in symmetric bilinear form, and λ denotes the characteristic function, calculated using extractions from exemplars with reference objects of interest known (see D. C. Brody and L. P. Hughston, “Geometry of Quantum Statistical Inference”, Physics Review Letters, 77(14), pp. 2851-2855, 1996).

Determination of Entrainment States

The OF derivation uses results from Daido's theory of multibranch entrainment of coupled nonlinear oscillators, wherein a number of different entrained states co-exist. For exemplars, an a priori measurement of entrainment is performed. This is done by using an approximate Daido Integral( ) with Zₖ=PSD maxima at regions where boundary of event of interest>desired detection threshold (see Physics Review Letters, 77(7), 1406-1411.) Pre-multiplier constants are used to ensure that Zₖ meets the following tests:

maxima power spectrum density (PSD) matches; and

L₂ norm on even harmonics <e where e=NS-MRF Barrier assuming complete spatial randomness (CSR) assumptions discussed in U.S. Pat. No. 6,136,541, entitled “Method and Apparatus for Analyzing Hybridized Biochip Patterns using Resonance Interactions Employing Quantum Expressor Functions all of which is hereby fully incorporated by reference”. Note that NS-MRF refers to the nonstationary Markov random field representation.

The notion of entrainment states is exploited, in part, because the method treats the spectral system embodied in an extraction core corresponding to the event of interest, as a special case of coupled nonlinear oscillators in equilibrium. However, due to device imperfections, signal degradation and other limitations, number of entrained binding states (i.e., background and clutter conditions) may coexist. A PSD match is desired where the OF is Z-peaked (but single peaked around each event of interest centroid). Absence of a single peak implies imperfect or lack of presence of an event of interest. It also defines the resonance loci for this method (i.e., where maximum SNR enhancement) is obtained.

Ground Truth based Modulation of OF

The OF of ground truth is modulated to yield the QEF as follows. Under controlled calibration, as stated above, maximal SNR enhancement (optimal resonance) is achieved when OF yields a single peak. It is an important design point for matching PSD of coupling Spin Boson system to the synthetic QEF. The specific form of the QEF to be used is the parameters of a generic OF shown above. So the exemplary method exploits two connotations of OF: (a) parametric form for the QEF (that is closer to the classical form) and (b) as exponential attractor for a dissipative system. The two OF's are then recoupled and convolved with spectrally transformed exemplar used for constructing the QEF.

The resulting QEF is given by:

$${QEF}_{{MRC}_{i}} = \ddot{H} ( \ddot{θ} ) = - {\sum }_{j = {l}_{x}}^{{u}_{x}} {\sum }_{k = {l}_{y}}^{{u}_{y}} {{h}^{⋒}}_{k \vert {υ}_{l = 1 , 2 , 3}} {Z}_{k} {ⅇ}^{- 2 π ik θ}$$

Typically, only first three to twenty-five PSD peaks are considered for spectral matching.

Preferably the QEF is represented digitally using a matrix or array having the same number of elements in the spectrally transformed and preconditioned, extraction core to be analyzed. In the exemplary embodiment this pertains to the preprocessing done to the post-hybridization microarray intensity output to get it into a form where it can be convolved with the Quantum Expressor Function.

A major limiting restriction in QSR that is avoided by the exemplary method pertains to matching the stochastic and deterministic time scales in “domain system” and the external coupling asymmetric dynamical system, since this has limited applicability to continuous data. By replacing the time scale match requirement with ensemble spatial statistics (statistical mechanics of the signal emitters responsible for generating the pattern of interest) expressed via the aforementioned generators an entirely new class of analysis for discrete systems is enabled. By deriving the condition that signal emitters need to satisfy to maximize QEF generator capacity, a properly constructed and conditioned QEF will be able to extract and enhance an entire class of interest to produce one or more expresser functions represented mathematically as

Q⁰:\overline{q}(q₁, q₂, . . . , qₚ)

b. Preconditioning

The signal pattern output by platform array detector 102 is represented mathematically as:

I(N,M)

Using the input signal pattern, the Preconditioner unit 104 performs the mathematical calculations detailed in U.S. patent application Ser. No. 10/430,664 entitled “Method and System for Characterizing Microarray Output Data” filed on May 5, 2003 which is hereby incorporated by reference, to generate the preconditioned output signal pattern represented mathematically as:

Ω(N,M).

c. Convolution Coupling

Active interferometric coupler 106 performs the following mathematical calculations using the preconditioned signal pattern:

\overline{ƒ}⁽⁰⁾

is defined as a vector containing the preconditioned components from an event of interest, and

\overline{ƒ}⁽ⁱ⁾=\overline{ƒ}⁽ⁱ⁻¹⁾\overline{Q}⁽ⁱ⁾

where Q⁽ⁱ⁾ represents is the QEF after i convolutions.

Thus

\overline{ƒ}⁽¹⁾=\overline{ƒ}⁽⁰⁾\overline{Q}⁽¹⁾

\overline{ƒ}⁽²⁾−\overline{ƒ}⁽¹⁾\overline{Q}⁽²⁾=\overline{ƒ}⁽⁰⁾\overline{Q}⁽²⁾\overline{Q}⁽¹⁾

where \overline{Q}⁽⁰⁾ represents the QEF developed in the preceding step and (its dimensionless quantity) and where \overline{Q}⁽ⁱ⁾ represents the i-th perturbation to the QEF, induced by perturbing one of its spectral components.

for k=1 to n

for j=1 to 1000 (set to a large counter value)

perturb the k^{th} component of QEF as below

Q^{j}(k)=[Q^{j-1}(k)+jC₁ sin(w₀j+C₁)]⁺

where

$${[ x ]}^{+} = \{ \begin{matrix}x & if & x \geq 0 \\ 0 & if & x < 0\end{matrix} \}$$

$$and$$

$${C}_{1} = \frac{1}{3} ( \frac{2 α}{360} ) ;$$

□ denotes a small constant;

Let W₀=the variance computed from the values

$$\frac{{\overline{f}}_{pc}}{Max ( {\overline{f}}_{pc} )}$$

where f_{pc} denotes the preconditioned spectral vector corresponding to a known event of interest present in the arrayed pattern being analyzed.

As an example, f_{pc} refers to the spectral components of the positive control.

The convolution iteration can be expressed as:

$${R}_{k j} = \frac{{\overline{f}}^{( j - 1 )} \cdot {\overline{Q}}^{j}}{{f}_{n c}^{( j - 1 )} \cdot {\overline{Q}}^{j}}$$

where f_{nc} refers to the spectral components of a canonical negative control, or preconditioned footprint of an event of interest known to be absent in the arrayed image.

After each convolution iteration check for monotonicity of

$$\frac{{R}_{k j + 2}}{{R}_{k j + 1}} > 1 AND \frac{{R}_{k j + 1}}{{R}_{k j}} > 1$$

if yes → then exit loop to perform global QEF iterations (that means this particular

k component is important, i.e we are diverging from the negative control.)

If no → then continue

end j loop

end k loop

Global QEF iterations may be performed if monotonic divergence is detected between the preconditioned extraction core being analyzed and the canonical negative control, then the same convolution coupling operations are repeated for all the spectral harmonics. The global QEF iterations are provided by:

Set \overline{r}⁰=\overline{q}^{(j+2)} A

For m=1 to 25 (chosen to be a small count value)

Compute

\overline{r}^{m}=\overline{r}^{m-1}+(m+j)C₁ sin(ω₀(m+j)+C₁)+mC₂ sin(ω₁ₘ+C₂)

where ω₁ captures the variance of the components of

$$\frac{{\overline{f}}_{N c}}{Max ( {\overline{f}}_{N c} )}$$

and

$${C}_{2} = {C}_{1} + ϵ ( \frac{Parseval Avg from Pos . Con . PM}{Parseval Avg from Neg . Con . PM} )$$

where Parseval Avg. from Pos. Con. PM refers to the parseval number for a canonical event of interest known to be present, and Parseval Avg. from Neg. Con. PM refers to the parseval number for a canonical event of interest known to be absent.

and ε is chosen to be small, 0.0001.

Again after each coupler iteration compute the term

$${R}_{m} = \frac{( {\overline{f}}^{j + m - 1} ) \cdot ( {\overline{r}}^{m} )}{( {\overline{f}}_{N c}^{j + m - 1} ) \cdot ( {\overline{r}}^{m} )}$$

Successively compute Rₘ, Rₘ₊₁, Rₘ₊₂ . . .

After each convolution iteration check if

$$\frac{{R}_{m + 2}}{{R}_{m + 1}} > 1 AND \frac{{R}_{m + 1}}{{R}_{m}} > 1$$

If the conditions of the above test are met, resonance is concluded and event of interest is called present.

If the monotonicity test fails, them the preconditioned test pattern is normalized using the expressions below.

if \overline{ζ}^{(j)}−\overline{ζ}^{(j-i)}>\overline{ζ}_{pc}^{(g)}−\overline{ζ}_{Nc}^{(c)} for any component then

$${\overline{f}}^{( j )} = {\overline{f}}^{( j )} - ( \frac{\sum {\overline{f}}^{( j )} - \sum {\overline{f}}^{( j - 1 )}}{25} )$$

The above assumes an object with 25 spectral harmonics of interest.

The detailed equations for the coupler unit and resonance detector unit are given below:

for j=1 to N

$$Δ j \to j + 1 = \frac{\sum ( {f}_{i} \cdot {\hat{Q}}^{j + 1 , p m} / \sum ( N C {F}_{i}^{0 , p m} \cdot {Q}^{j + 1} )}{\sum ( {f}_{i}^{o , p m} \cdot {Q}^{j , p m} ) / \sum ( N C {F}_{i}^{g , p m} \cdot {Q}^{j , p m} )}$$

$$\begin{matrix}{Q}^{j + 1} \\ j \neq k\end{matrix} = {\hat{Q}}^{j}$$

$$\begin{matrix}{Q}^{j + 1} \\ i = k\end{matrix} = [ \begin{matrix}{\hat{Q}}^{j} \\ i = k\end{matrix} + ( {φ}_{c} \cdot j ) sin ( {w}_{0} j + {φ}_{c} ) ]$$

$$Check \frac{{Δ}_{j - 1 , j + 2}^{2}}{Δ j} > 1$$

$$\frac{Δ j + 1 \to j + 2}{Δ j \to j + 1} > 1 \vert \frac{Δ j \to j + 1}{Δ j \to j} > 1$$

$${φ}_{c} = \frac{1}{3} \cdot \frac{2 π}{360}$$

QEFᵣ₊₁={QEFᵣ+A(j,r)+B/r}

where B=ζ(w₁) and

$${W}_{1} = σ ( \frac{\sqrt{{PSD}_{pm , njc}}}{\sqrt{man {PSD}_{pm , njc}}} )$$

A=φ_{c}(j+r)·{sin (wₒ(j+r)+φ_{c})}

B=φ_{c1}r)·{sin (w,(r)+φ_{c1})}

$${φ}_{{c}^{1}} = {φ}_{c} + ϵ ( \frac{{σ}_{1}}{{σ}_{2}} ) \{ = 0.0001 \}$$

$${W}_{0} = σ ( \frac{P S {D}_{Q E F p c , p m}}{max ( P S {D}_{Q E F ; p c , p m} )} )$$

$${W}_{1} = σ ( \frac{P S {D}_{Q E F N C , p m}}{max ( P S {D}_{Q E F , N C , p m} )} ) {w}_{0} \approx {w}_{1}$$

d. Resonant Marker Identification

Finally, resonance marker detector 108 performs the following mathematical calculations using the convolved signal pattern to identify the events of interest within the convolved signal pattern.

The resonant iteration is terminated when

$$\begin{matrix}\frac{{Δ}_{j - 1 , j + 2}^{2}}{Δ j} > 1 \\ \frac{Δ j + 1 \to j + 2}{Δ j \to j + 1} > 1 \vert \frac{Δ j \to j + 1}{Δ j \to j} > 1\end{matrix}$$

or when iteration counter t exceeds preset “N” (e.g., 10³ iterations) (for digital approximation to analog dynamics).

Preferably, the resonance interaction is performed digitally by applying a matrix representative of the resonance equation to a matrix representative of the resonance stimulus in combination with a matrix representative of the preconditioned extraction of event of interest.

The final result of interferometric analysis is the readout of locations wherein resonance has occurred and as identified by row and column number (i,j).

Resonance Output Interpretation to Identify Gene Expression

For example, with regard to gene expression, once resonances peak are observed by applying a QEF to specific object extractions, the object addresses (k,l) of those locations are mapped into the physical feature or gene table (which contains the complimentary RNA or cDNA probe sequences associated with object locations) to thereby identify the gene being expressed, if any, present in the sample being analyzed. This is a simple table look-up resulting in a direct readout of the names of the expressed genes.

To summarize the open loop interferometric technique of FIGS. 1 and 2, the preconditioning of arrayed data to the spectral domain is performed independently prior to coupling with the expressor function. Hence, the reverberation convergence performed by the active interferometric coupler to achieve a resonance state is performed independent of preconditioning. The reverberations are an open loop process terminated upon detection of a predetermined condition (i.e. observation of a resonance marker.) The active interferometric coupler implements either a destructive or a constructive interference of two dynamical systems.

B. Goal-Directed Interferometric System

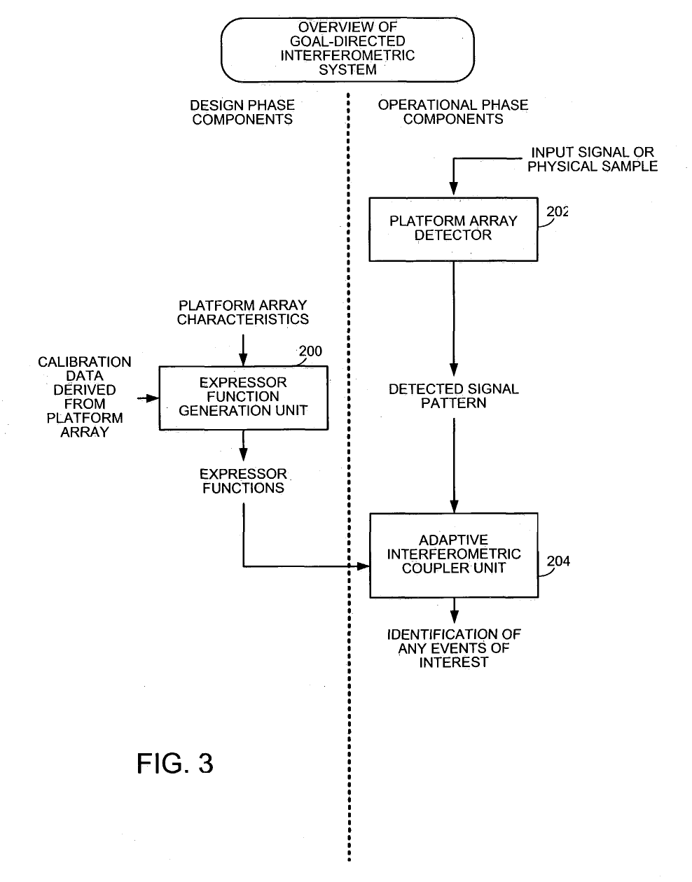

FIG. 3 illustrates exemplary system components for the second exemplary implementation of the invention. As before, components employed only during a design phase are shown on the left and components employed during the actual analysis of an input signal are shown on the right. Some of the components of the system of FIG. 3 are the same as those shown in FIG. 1 and hence will be not described again in detail. Again, during a design phase, an expresser function generation unit 200 generates expressor functions based upon calibration data and based on the specific characteristics of a platform array detector 202 and during the operational phase, the platform array detector generates a signal pattern for analysis. However, no separate preconditioner unit is provided. Nor is any separate resonant maker detector provided. Rather, a single adaptive interferometric coupler 204 is provided that operates to simultaneously precondition the detected signal pattern to convert the arrayed signal pattern to a spectral domain while convolving the signal pattern and the expressor functions to interferometrically enhance of portions of the signal pattern and to identify events of interest, if any, within the preconditioned signal pattern. Again, with the exception of the platform array detector, which is a physical hardware component, all other components illustrated in FIG. 3 may be implemented in software, hardware, firmware or some combination thereof

Note that adaptive interferometric coupler 204 does not merely perform the operations previously described with respect components 104, 106 and 108 of FIG. 1. Rather, adaptive interferometric coupler 204 performs a different set of operations. Briefly, with the technique of FIG. 3, coupling intelligence is merged with preconditioning and the expressor functions are pre-coupled to the preconditioned functions, as will be apparent from the detailed example below. Convergent reverberation is performed simultaneously with preconditioning, which is now a closed loop process. Hence, with the technique of FIG. 3, preconditioning does not merely transform spatial arrayed data to spectral data but also transforms the data to a pre-determined state of a predetermined dynamical system. If that state is reached, then resonance is implicitly assumed to have occurred. Thus, completion of the adaptive interferometric coupling procedure implies the detection of resonant markers and so separate resonant marker detection unit is not required.

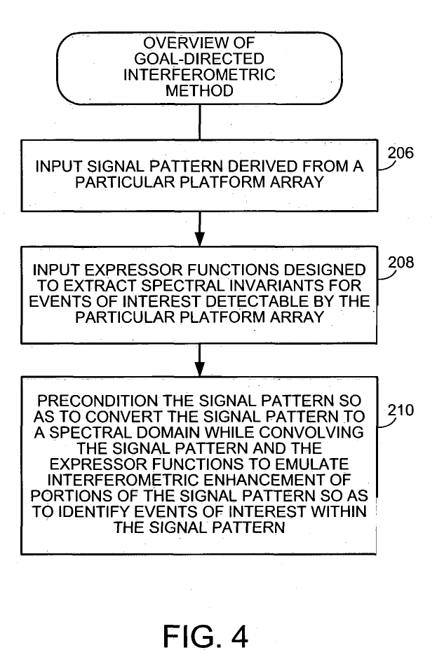

Method steps associated with the goal-directed interferometric system will now briefly be summarized with reference to FIG. 4.. At step 206, a signal pattern derived from a particular platform array detector is input and, at step 208, expressor functions designed to extract spectral invariants for events of interest detectable by the particular platform array detector are input. Then, at step 210, the input signal pattern is preconditioned to convert the signal pattern to the spectral domain while convolving the signal with the expresser functions so as to emulate an active interferometric enhancement of portions of the signal pattern so as to identify events of interest therein. As with the technique described above, a list of specific events of interest need not be input during the operational phase. Rather, the technique operates to detect possible events of interest present in the input pattern based solely on the input pattern and the previously-generated expresser functions.

One of ordinary skill in the art will appreciate that the equations provided above in connection with the open loop interferometric system may be accordingly adjusted such that the resonant marker unit and the convolution coupling unit may be combined so that the threshold check for resonance is performed during the post-convolution normalization.

C. Self-Organizing Interferometric System

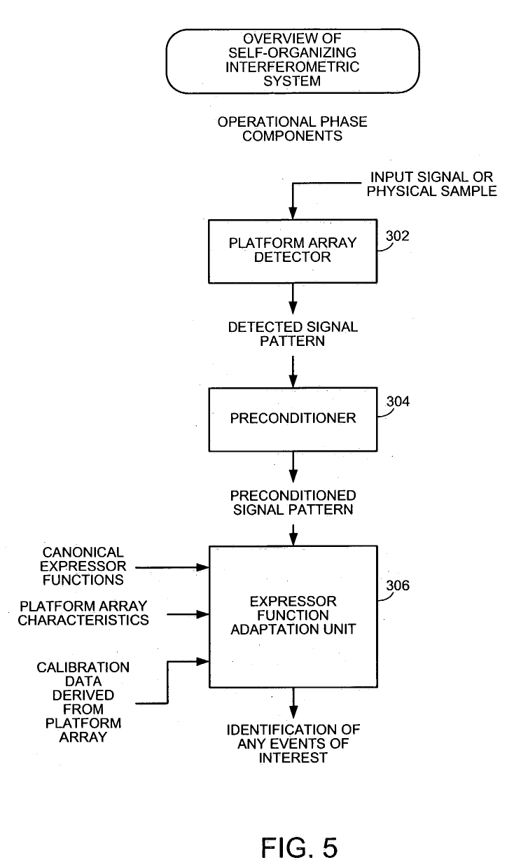

FIG. 5 illustrates exemplary system components for the third exemplary implementation of the invention. Unlike the systems thus far described, no separate design phase is required. Rather, all components operate during the actual analysis of an input signal. Again, some components are the same as those described above and hence will be not described again in detail. In use, a platform array detector 302 generates a signal pattern for analysis. The signal pattern is preconditioned by a preconditioner unit 304, which, as before, operates to convert the signal pattern to a spectral domain in which spectral harmonics parameterize events of interest to a predetermined dynamical system. The preconditioned signal pattern as well as characteristics of the platform array detector, calibration data and a set of canonical expressor functions are then all input by an expressor function adaptation unit 306. The canonical expresser functions are generalized expressor functions that have not been tailored to the particular platform being used. No separate design phase expresser function generation unit is provided. Nor is a separate resonant maker detector provided. Rather, expresser function adaptation unit 306 operates to simultaneously generate preconditioned expresser functions based on the preconditioned signal pattern and based on the canonical expressor functions while convolving the preconditioned signal pattern and the preconditioned expresser functions to interferometrically enhance portions of the signal pattern to identify events of interest. As before, with the exception of the platform array detector, which is a physical hardware component, all other components illustrated in FIG. 5 may be implemented in software, hardware, firmware or some combination thereof.

Note that expressor function adaptation unit 306 does not merely perform the operations previously described with respect to components 100, 106 and 108 of FIG. 1. Rather, with the technique of the self-organizing interferometric system, the coupling intelligence and the reverberation dynamics are pre-built into the expresser functions and so the expresser functions are not merely a representation of a spectral marker as with the technique of FIGS. 1 and 2 and with the technique of FIGS. 3 and 4. This will be more readily apparent from the detailed example below. Also, although the preconditioning unit may be the same as in the technique of FIGS. 1 and 2, the preconditioned array now serves as a preconditioner to the expresser function to thereby produce a properly preconditioned expressor, which itself corresponds to the detection of resonant marker. Hence, generating and outputting the properly preconditioned expresser function results in detection of the event of interest. As the expresser functions are tailored to the preconditioned array, they cannot be used again to process other signal patterns detected by the platform array detector. Also, as will be described in greater detail below, with the technique of the self-organizing interferometric system, the operations of the expressor function adaptation unit operate in an internal closed loop.

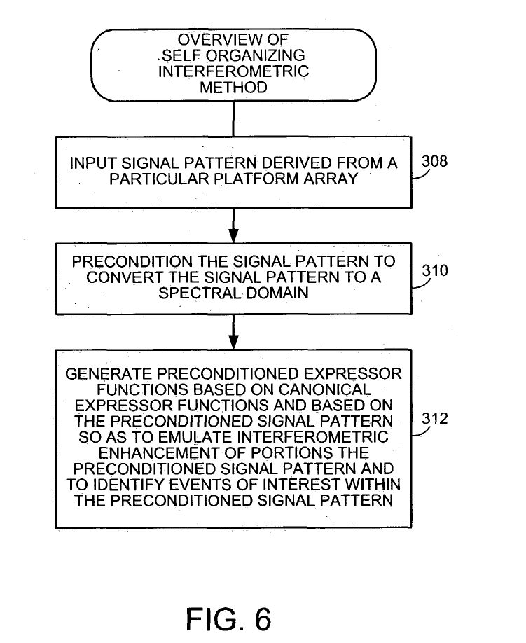

Method steps associated with the self-organizing interferometric system will now briefly be summarized with reference to FIG. 6. At step 308, a signal pattern derived from a particular platform array detector platform is input and, at step 310, the signal patterns are preconditioned to convert the signal pattern to the spectral domain in which spectral harmonics parameterize events of interest to the aforementioned predetermined dynamical system. At step 312, preconditioned expressor functions are then generated based on the preconditioned signal pattern and on the characteristics of the platform array detector and based on the events of interest. This is performed using a closed loop process employing reverberant convergence so as to emulate active interferometric enhancement of portions of the original signal pattern associated with the events of interest. Hence, generation of the preconditioned expressor functions allows immediate detection of the events of interest without further processing. As before, no list of specific events of interest need be input during the operational phase.

One of ordinary skill in the art will appreciate that the equations provided above in connection with the open loop interferometric system may be accordingly adjusted such that the modulation of the OF to generate a QEF is immediately succeeded by the convolution coupling.

D. Iterative Interferometric System

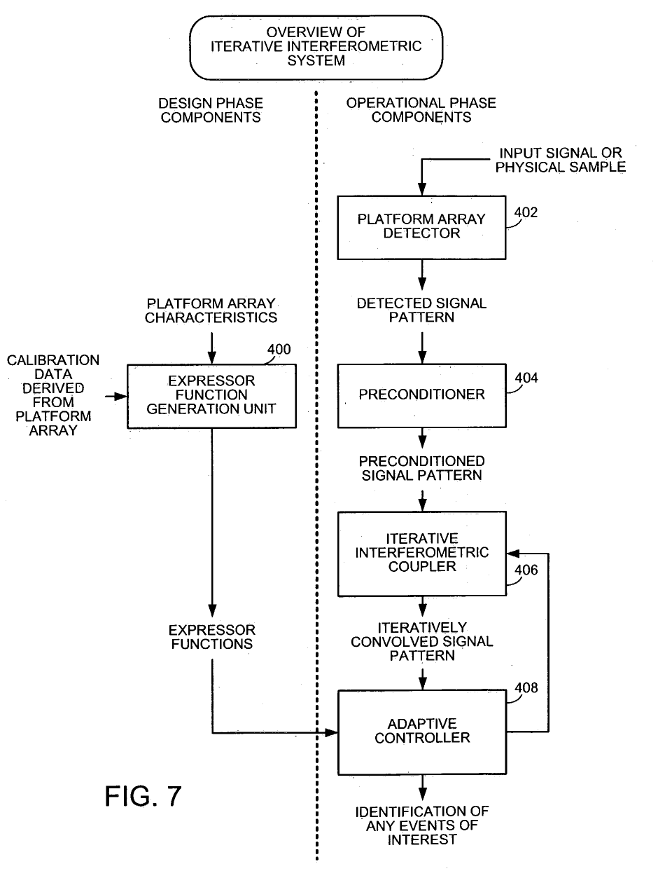

FIG. 7 illustrates exemplary system components for the fourth exemplary implementation of the invention. As before, components employed only during a design phase are shown on the left and components employed during the actual analysis of an input signal are shown on the right and components already described will be not described again in detail. During the design phase, an expresser function generation unit 400 generates expresser functions based upon calibration data and based on the specific characteristics of a platform array detector 402 and, during the operational phase, the platform array detector generates a signal pattern for analysis. The signal pattern is preconditioned by a preconditioner unit 404, which, as before, operates to convert the signal pattern to a spectral domain in which spectral harmonics parameterize events of interest to a predetermined dynamical system. An iterative interferometric coupler 406 convolves the preconditioned signal pattern and the previously-generated expresser functions so as to interferometrically enhance portions of the preconditioned signal pattern associated with events of interest that are present within the preconditioned signal pattern. However, unlike the technique of the open loop interferometric system of FIGS. 1 and 2, wherein the convolution coupler is a stand-alone unit that operates to generate a final convolved signal pattern, with the technique of FIG. 7, the iterative interferometric coupler operates under the control of an adaptive controller 408 to sequentially produce an iteratively convolved signal pattern. As explained more fully below, the adaptive controller operates to control the interferometric coupler to iteratively and selectively convolve expressor functions to the preconditioned signal pattern until a predetermined degree of convergence is achieved so as to allow identification of events of interest within the enhanced signal pattern. Again, with the exception of the platform array detector, all other components illustrated in FIG. 7 may be implemented in software, hardware, firmware or some combination thereof.

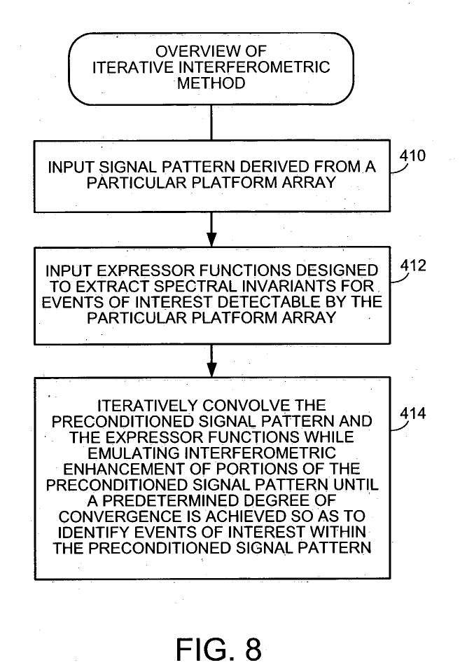

Method steps associated with the iterative interferometric system will now briefly be summarized with reference to FIG. 8. At step 410, a signal pattern derived from a particular platform array detector is input and, at step 412, expresser functions designed to extract spectral invariants for events of interest detectable by the particular platform array detector are input. Then, at step 414, the input signal pattern is preconditioned to convert the signal pattern to the spectral domain spectral domain in which spectral harmonics parameterize events of interest to the aforementioned predetermined dynamical system. At step 416, the preconditioned signal pattern and the expresser functions are then iteratively convolved under the control of the controller to achieve a reverberant convergence so as to emulate active interferometric enhancement of the signal pattern associated and to allow detection of events of interest. As before, no list of specific events of interest need be input during the operational phase.

One of ordinary skill in the art will appreciate that the equations provided above in connection with the open loop interferometric system may be accordingly adjusted such that multiple QEFs may be employed to achieve a desired resonance level and the most relevant QEF is chosen after each convolution if a desired resonance is not achieved.

E. General Features

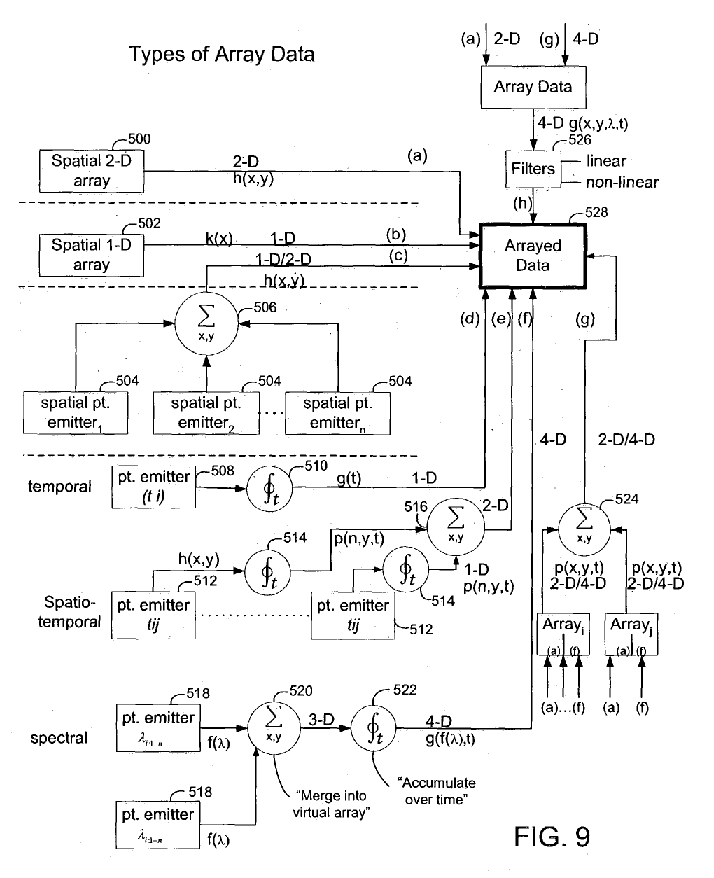

Exemplary embodiments are described above wherein arrayed data generated by a platform array detector is processed to identify events of interest therein. A wide variety of types of arrayed data maybe processed. FIG. 9 lists specific examples of types of arrayed data that may be processed. The examples shown include spatial 2-D data, spatial 1-D data, point emitter data, temporal point emitter data, spatio-temporal point emitter data and spectral point emitter data. The different types of arrayed data may be separately processed or, as shown, combined to yield virtual arrays for processing. In FIG. 9, the symbol ψ generally represents a virtual array combiner operation, wherein individual spatial values are collected together into a single function or data array. For example, if sixteen individual point emitters are provided, the data from the sixteen point emitters is combined or merged to yield a single 4×4 virtual array (represented, e.g., as f(x,y)). The symbol ι generally represents a temporal accumulation operation, wherein individual values are accumulated over time to yield a single time-varying function or data array. For example, if a single point emitters is provided, the point emitter may be sampled periodically to accumulate values to populate a time-varying function (represented, e.g., as f(t)). In some cases, both virtual array and accumulation operations are performed to generate spatio-temporal data (represented, e.g., as f(x,y,t)).

More specifically, FIG. 9 illustrates:

(a) spatial data from a 2-D spatial array 500 represented in the form of a 2-D function h(x,y);

(b) spatial data from a 1-D spatial array 502 represented the form of a 1-D function k(x);

(c) spatial data from a group of point emitters 504 (with each point emitters, for i=1 . . . n, providing a single non-time-varying value) assembled into a single 2-D function h(x,y) via a virtual array combiner 506;

(d) temporal data from a single point emitter 508 accumulated over time via a temporal accumulator 510 to yield a single 1-D function g(t);

(e) spatio-temporal data from a group of individual point emitter arrays 512 (with each point emitterᵢ, for i=1 . . . n, providing a 2-D array non-time varying values) each separately accumulated over time via temporal accumulators 514 to yield individual functions p(n,y,t), which are in turn assembled into a 2-D spatio-temporal function via a virtual array combiner 516; and

(f) spectral data from a group of individual spectral point emitters 518 (with each spectral point emitterᵢ, for i=1 . . . n, providing an output function f(λ) varying with wavelength) assembled into a 3-D virtual spectral array via virtual array combiner 520, with values then accumulated over time via temporal accumulators 522 to yield a 4-D function g(x,y,λ,t).

Additionally, the various types of arrayed data ((a)-(f)) may be further combined into a larger virtual array (g) by virtual array combiner 524. Also, any of the types of arrayed data ((a)-(g)) may be further filtered via linear or non-linear filters 526, to yield filtered array functions (h). The final collection of data is represented in the figure as arrayed data 528. As can be appreciated, a wide variety of arrayed data may be input, combined and manipulated to yield individual multi-dimensional input functions for processing by the above-described systems.

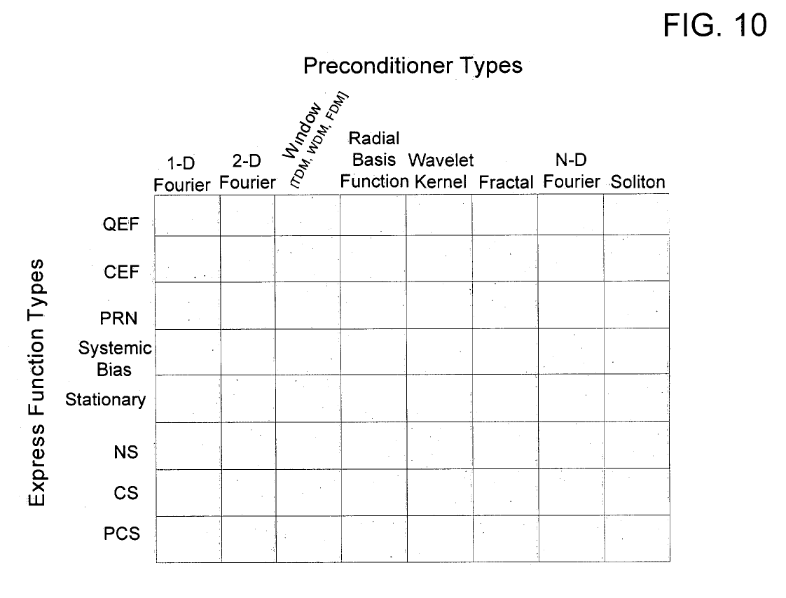

Also, a wide variety of preconditioner functions may be employed for use in preconditioning the arrayed data and a wide variety of expresser functions may be generated for use in processing the preconditioned arrays. FIG. 10 lists specific examples of types of expresser functions and types of preconditioning functions. The types of expressor functions are identified in terms of the type of noise associated with the expressor function. Examples of expresser functions include quantum expressor functions (QEFs), classical expressor functions (CEFs), classical statistical noise expresser functions (SCNEFs), pseudorandom noise expressor functions (PRNEFs), systemic bias expresser functions and expressor functions based on polycyclostationary noise (PCS), cyclostationary noise (CS), stationary noise (SN), non-stationary noise (NS). Examples of preconditioner functions include 1-D Fourier functions, 2-D Fourier functions, N-D Fourier functions, a time division multiplexing (TDM) functions, wavelength division multiplexing (WDM) functions, frequency division multiplexing (FD) functions, radial basis functions, wavelet kernel functions, fractal functions and soliton functions. In general, any combination of expressor function type and preconditioner function type illustrated in the figure may be employed.

FIG. 11 illustrates an exemplary system 700 incorporating a biological platform array 702 that provides arrayed data to a software-based active interferometric analysis system 704. The platform array includes a solid support 706 upon which various probes 708 are attached via attachment chemistry portion 710 in the example of FIG. 11, the probes include one or more of cDNA, mRNA or oligonucleotide probes or proteins, peptides or oligopeptides. Following application of a biological sample, such as blood or urine, to the probes, a detector 712 detects a resulting pattern, which is then forwarded to the analysis system in the form of arrayed data. The analysis system exploits one of the general techniques described above to identify events of interest present in the biological sample. In this example, the events of interest are typically output in the form of one or more “gene calls”.

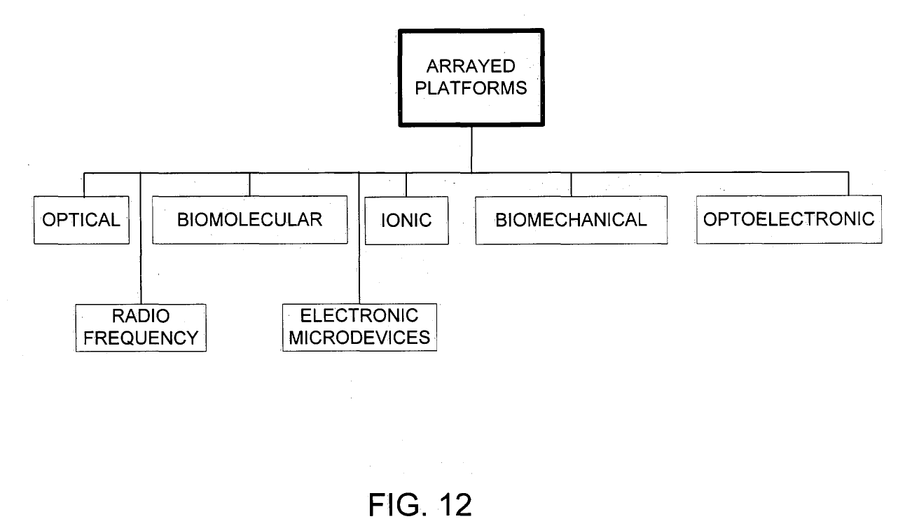

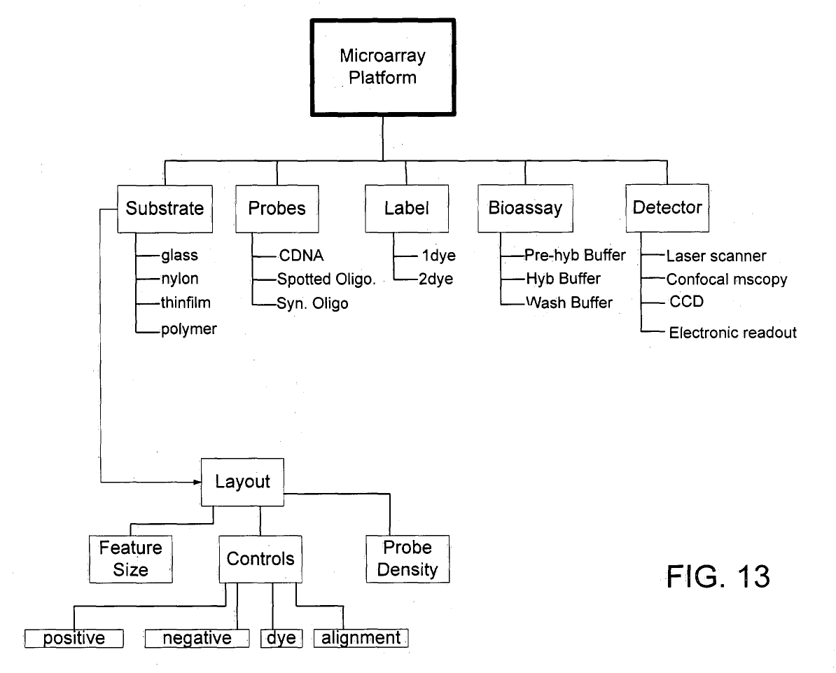

A wide variety of different platform array detectors may be used to generate the arrayed data. Examples of platforms detectors are listed in FIG. 12. The examples include optical platforms, biomolecular platforms, ionic platforms, biomechanical platforms, optoelectronic platforms, radio frequency platforms, and other electronic microdevices. Exemplary biomolecular spatial array platforms include: hybridized spotted cDNA microarrays, synthesized oligonucleotide arrays, spotted oligonucleotide arrays, peptide nucleotide assays, single nucleotide polymorphism (SNP) arrays, carbohydrate arrays, glycoprotein arrays,. protein arrays, proteomic arrays, tissue arrays, antibody arrays, antigen arrays, bioassays, sequencing microarrays, sequencing by hybridization (SBH) microarrays, siRNA duplexes, RNAi arrays glass-based arrays, nylon membrane arrays, thin film arrays, polymer-substrate arrays, capillary electrophoresis arrays, genospectral arrays, electronic arrays, bead arrays, quantum dot arrays, glycan arrays, spotted wells, and spotted well plates. Typically, any of the foregoing platforms may be implemented as a microarray. Components of a typical microarray are shown in FIG. 13, which include a substrate, a probe portion, a label portion, a bioassay portion, and a detector. Exemplary substrates include glass, nylon, thin film, and polymer. Exemplary probes cDNA probes, spotted oligonucleotide probes and synthetic oligonucleotide probes. Exemplary labels 1-dye labels and 2-dye labels. Exemplary bioassay portions include prehybridization buffers, hybridization buffers and wash buffers. Exemplary detectors include laser scanners, confocal microscopy detectors, charge coupled devices (CCDs) and electronic readouts. The substrate has a certain layout represented by feature size, various controls (positive, negative dye and alignment), and probe density.

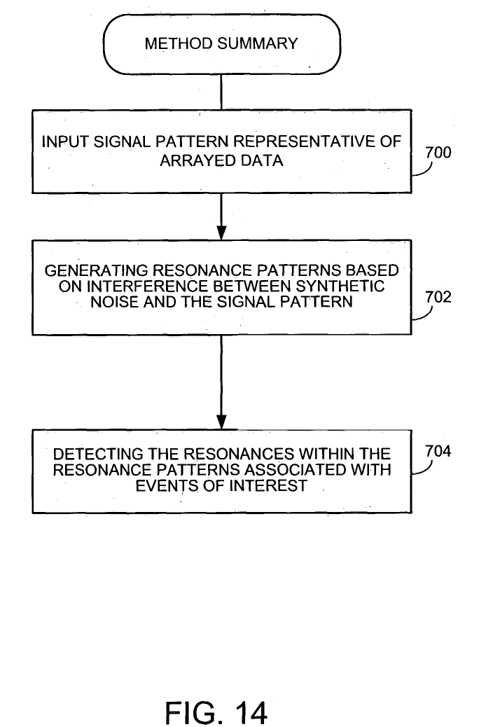

FIG. 14 provides a summary of an exemplary technique of the invention. Briefly, the figure illustrates method for actively analyzing a signal pattern representative of arrayed data to identify events of interest therein. At step 700, a signal pattern representative of arrayed data is input and, at step 702, resonance patterns are generated based on interference between synthetic noise and the signal pattern, in accordance with techniques already described. Thereafter, at step 704, resonances are detected within the resonance patterns associated with events of interest. The synthetic noise used at step 702 is preferably in the form of a quantum expresser function, a classical expresser function, classical statistical noise, pseudorandom noise, a systemic bias or some combination thereof The arrayed data may be, as already explained, in the form of a spatial 2-D array, a spatial 1-D array, an N-D array, a temporal point emitter array, a spatio-temporal point emitter array, spectral point emitter array or a virtual array constructed by combining spatial separate point emitters.

The description of the exemplary embodiments is provided to enable any person skilled in the art to make or use the present invention. Various modifications to these embodiments will be readily apparent to those skilled in the art and the generic principles defined herein may be applied to other embodiments without the use of the inventive faculty. Thus, the invention is not intended to be limited to the embodiments shown herein but is to be accorded the widest scope consistent with the principles and novel features disclosed herein.

What is claimed is:

1. A system for performing active interferometric analysis using an expresser function to detect the presence of an event of interest within an arrayed signal pattern via a computationally induced interference mechanism.

2. The system of claim 1 implemented in software.

3. The system of claim 1 wherein the arrayed signal pattern comprises either static data or dynamic data.

5. The system of claim 1 wherein the expresser function is a quantum expressor function.

6. The system of claim 1 wherein the arrayed signal pattern is in the form of one or more of a spatial 1-D array, a spatial 2-D array, an N-D array, a temporal point emitter array, a spatio-temporal point emitter array, spectral point emitter array, or a virtual array constructed by combining spatial separate point emitters.

7. The system of claim 1 wherein the arrayed signal pattern is generated from a device chosen from the group comprising: optical platforms, biomolecular platforms, ionic platforms, biomechanical platforms, optoelectronic platforms, radio frequency platforms, electronic microdevices.

8. The system of claim 1 wherein the arrayed signal pattern is generated from a device chosen from the group comprising: hybridized spotted cDNA microarrays, synthesized oligonucleotide arrays, spotted oligonucleotide arrays, peptide nucleotide assays, single nucleotide polymorphism (SNP) arrays, carbohydrate arrays, glycoprotein arrays, protein arrays, proteomic arrays, tissue arrays, antibody arrays, antigen arrays, bioassays, sequencing microarrays, sequencing by hybridization (SBH) microarrays, siRNA duplexes, RNAi arrays glass-based arrays, nylon membrane arrays, thin film arrays, polymer-substrate arrays, capillary electrophoresis arrays, genospectral arrays, electronic arrays, bead arrays, quantum dot arrays, glycan arrays, spotted wells, spotted well plates.

9. The system of claim 1 wherein the expressor function is designed to reject any interfering noise and background clutter from events of interest.

10. The system of claim 9 wherein the expressor function is designed so as to extract spectral invariants of events of interest associated with an array platform device used to detect the arrayed signal pattern.

11. The system of claim 1 wherein the expressor function is comprised of frequency domain sequences, time domain sequences or spectral sequences, phase sequences or numeric sequences.

12. A system for performing active interferometric signal analysis of static data from an arrayed platform in software.

13. The system of claim 12 wherein the active interferometric analysis includes detection and quantitation analysis.

14. The system of claim 12 wherein the active interferometric analysis includes constructive interferometric analysis.

15. The system of claim 12 wherein the active interferometric analysis includes destructive interferometric analysis.

16. The system of claim 12 wherein the active interferometric analysis includes software emulation of wave-particle interactions.

17. The system of claim 12 wherein the active interferometric analysis includes software emulation of wave-wave interactions.

18. The system of claim 12 wherein the active interferometric analysis includes one or more of frequency domain, time domain, or phase domain analysis.

19. The system of claim 12 wherein the active interferometric analysis exploits iterative convergence to detect resonance events.

20. A system for performing active interferometric analysis by using reverberant convergence to detect resonance events.

21. The system of claim 20 exploiting quantum interferometric analysis.

22. The system of claim 21 wherein the quantum interferometric analysis is applied to one or more of a static spatial system, a static data from arrayed measurement platforms, dynamical systems, spatio-temporal systems and plasma systems.

23. The system of claim 20 wherein the active interferometric analysis exploits non-classical noise.

24. The system of claim 23 wherein the active interferometric analysis exploits one or more of non-Gaussian noise, ergodic noise, and quantum-mechanical noise.

25. The system of claim 20 wherein the active interferometric analysis exploits resonant interferometric signal analysis using a digital representation of spectral pulses.

26. The system of claim 20 wherein the active interferometric signal analysis exploits one or more of coherent, incoherent, synchronized, or asynchronous oscillations.

27. The system of claim 20 wherein the active interferometric analysis exploits time domain or frequency domain convolution operations.

28. The system of claim 20 wherein the active interferometric analysis exploits expressor functions to detect the presence of an event of interest via a computationally induced interference mechanism.

29. A method for actively analyzing a signal pattern representative of arrayed data to identify events of interest therein, the method comprising the steps of:
inputting a signal pattern representative of arrayed data;
generating resonance patterns based on interference between synthetic noise and the signal pattern; and
detecting resonances within the resonance patterns associated with events of interest.

30. The method of claim 29 wherein the synthetic noise is in the form of one or more of a quantum expressor function, a classical expressor function, classical statistical noise, pseudorandom noise, or a systemic bias.

31. The method of claim 29 wherein the arrayed data is in the form of one or more of a spatial 2-D array, a spatial 1-D array, an N-D array, a temporal point emitter array, a spatio-temporal point emitter array, spectral point emitter array or a virtual array constructed by combining spatial separate point emitters.

32. The method of claim 29 further including the step of pre-conditioning the signal pattern prior to the step of generating resonance patterns.

33. The method of claim 32 wherein the step of pre-conditioning the signal pattern is performed by applying one or more preconditioning functions in the form of a 1-D Fourier function, a 2-D Fourier function, an N-D Fourier function, a time division multiplexing (TDM) function, a wavelength division multiplexing (WDM) function, a frequency division multiplexing (FD) function, a radial basis function, a wavelet kernel function, a fractal function, a soliton function.

34. The method of claim 29 wherein the arrayed data is generated from a device chosen from the group comprising optical platforms, biomolecular platforms, ionic platforms, biomechanical platforms, optoelectronic platforms, radio frequency platforms, electronic microdevices.

35. The method of claim 29 wherein the arrayed data is generated from a device chosen from the group comprising: hybridized spotted cDNA microarrays, synthesized oligonucleotide arrays, spotted oligonucleotide arrays, peptide nucleotide assays, single nucleotide polymorphism (SNP) arrays, carbohydrate arrays, glycoprotein arrays, protein arrays, proteomic arrays, tissue arrays, antibody arrays, antigen arrays, bioassays, sequencing microarrays, sequencing by hybridization (SBH) microarrays, siRNA duplexes, RNAi arrays glass-based arrays, nylon membrane arrays, thin film arrays, polymer-substrate arrays, capillary electrophoresis arrays, genospectral arrays, electronic arrays, bead arrays, quantum dot arrays, glycan arrays, spotted wells, spotted well plates.

36. A system for analyzing a signal pattern to identify events of interest within the signal pattern comprising:
an expressor function input unit;
a preconditioner unit;
an active interferometric coupler; and
a resonant marker detector.

37. A system for analyzing an arrayed signal pattern generated by an arrayed platform device to identify events of interest within the signal pattern comprising:
an expressor function input unit for inputting expressor functions;
a preconditioner unit for preconditioning the arrayed signal pattern so as to convert the arrayed signal pattern to a spectral domain in which spectral harmonics parameterize events of interest to a pre-determined dynamical system;
a coupler unit for convolving the preconditioned signal pattern and the expresser functions so as to interferometrically enhance portions of the preconditioned signal pattern associated with events of interest, if any, present within the preconditioned signal pattern; and
a resonant marker detector for identifying occurrence of events of interest within the enhanced signal pattern.

38. The system of claim 37 wherein the signal pattern is imaged and pixilated.

39. The system of claim 37 wherein the expressor functions are designed so as to be capable of extracting one or more of spatial, spatio-temporal or spectral invariants of events of interest associated with the array device.

40. A method for analyzing an arrayed signal pattern generated by an array device to identify events of interest within the signal pattern comprising the steps of:
inputting expressor functions capable of extracting spectral invariants of events of interest associated with the array device;
preconditioning the arrayed signal pattern so as to convert the arrayed signal pattern to a spectral domain;
convolving the preconditioned signal pattern and the expressor functions so as to interferometrically enhance portions of the preconditioned signal pattern associated with events of interest, if any, present within the preconditioned signal pattern; and
identifying events of interest within the enhanced signal pattern.

41. The method of claim 40 wherein the step of convolving the preconditioned signal pattern and the expressor functions includes the step of performing a reverberation convergence between the preconditioned signal pattern and the expressor functions to achieve a resonance state.

42. The method of claim 41 wherein the step of performing a reverberation convergence is performed using an open loop control process that terminates upon detection of a predetermined condition.

43. The method of claim 40 wherein the predetermined condition is a resonant marker corresponding to an event of interest.

44. The method of claim 40 wherein the step of convolving the preconditioned signal pattern and the expresser functions includes the step of a destructive interference between the preconditioned signal pattern and the expresser functions.

45. The method of claim 40 the step of convolving the preconditioned signal pattern and the expresser functions includes the step of a constructive interference between the preconditioned signal pattern and the expressor functions.

46. A system for analyzing a signal pattern to identify events of interest within the signal pattern comprising:
an expresser function input unit; and
an adaptive interferometric coupler operative to perform signal preconditioning, convolution coupling and resonant marker detection using the expresser functions.

47. A system for analyzing an arrayed signal pattern generated by an arrayed platform device to identify events of interest within the signal pattern comprising:
an expressor function input unit for inputting expresser functions capable of extracting spectral invariants of events of interest associated with the arrayed signal pattern; and
an adaptive interferometric coupler for preconditioning the arrayed signal pattern so as to convert the arrayed signal pattern to a spectral domain to a spectral domain in which spectral harmonics parameterize events of interest to a pre-determined dynamical system while convolving the signal pattern and the expresser functions so as to interferometrically enhance of portions of the signal pattern and to identify events of interest, if any, within the signal pattern.

48. A method for analyzing an arrayed signal pattern generated by an arrayed platform device to identify events of interest within the signal pattern comprising the steps of:
inputting expresser functions capable of extracting spectral invariants of events of interest associated with the array device; and
preconditioning the arrayed signal pattern so as to convert the arrayed signal pattern to a spectral domain to a spectral domain to a spectral domain in which spectral harmonics parameterize events of interest to a pre-determined dynamical system while convolving the signal pattern and the expressor functions so as to interferometrically enhance portions of the signal pattern and to identify events of interest within the signal pattern.

49. The method of claim 48 wherein the step of preconditioning the signal pattern includes the step of performing a closed loop reverberation convergence between the signal pattern, as it is being preconditioned, and the introduced expresser functions to achieve a resonance state, terminated upon detection of a predetermined condition.

50. The method of claim 48 wherein the predetermined condition is a resonant marker.

51. A system for analyzing a signal pattern to identify events of interest within the signal pattern comprising:
a preconditioner unit; and
an expresser function adaptation unit operative to perform convolution coupling and resonant marker detection using canonical expresser functions.

52. A system for analyzing an arrayed signal pattern generated by an arrayed platform device to identify events of interest within the signal pattern comprising:
a preconditioner for preconditioning the arrayed signal pattern so as to convert the arrayed signal pattern to a spectral domain to a spectral domain in which spectral harmonics parameterize events of interest to a pre-determined dynamical system; and
an expresser function adaptation unit for generating preconditioned expressor functions based on canonical expresser functions and based on the preconditioned signal pattern while extracting spectral invariants of events of interest associated with the array device and interferometrically enhancing portions the preconditioned signal pattern so as to identify events of interest, if any, within the signal pattern.

53. A method for analyzing an arrayed signal pattern generated by an array device to identify events of interest within the signal pattern comprising the steps of:
preconditioning the arrayed signal pattern so as to convert the arrayed signal pattern to a spectral domain to a spectral domain in which spectral harmonics parameterize events of interest to a pre-determined dynamical system; and
generating preconditioned expressor functions based on canonical expresser functions and based on the preconditioned signal pattern while extracting spectral invariants of events of interest associated with the array device and interferometrically enhancing portions the preconditioned signal pattern to identify events of interest, if any, within the signal pattern.

54. The method of claim 53 wherein the step of generating preconditioned expressor functions is performed so as to implicitly achieve a reverberation convergence between the preconditioned signal pattern and the expressor functions to achieve a resonance state.

55. The method of claim 53 wherein the step of generating preconditioned expressor functions is performed using a closed loop process.

56. A system for analyzing a signal pattern to identify events of interest within the signal pattern comprising:
an expressor function input unit;
a preconditioner unit;
an iterative interferometric coupler; and
an adaptive controller operative to control the iterative interferometric coupler to perform convolution coupling and resonant marker detection using the input expresser functions.

57. A system for analyzing an arrayed signal pattern generated by an arrayed platform device to identify events of interest within the signal pattern comprising:
an expressor function input unit for inputting expressor functions capable of extracting spectral invariants of events of interest associated with the array device;
a preconditioner for preconditioning the arrayed signal pattern so as to convert the arrayed signal pattern to a spectral domain to a spectral domain in which spectral harmonics parameterize events of interest to a pre-determined dynamical system;
an iterative interferometric coupler for convolving the preconditioned signal pattern and the expresser functions so as to interferometrically enhance portions of the preconditioned signal pattern associated with events of interest, if any, present within the preconditioned signal pattern; and
an adaptive controller for controlling the coupler to iteratively and selectively convolve expressor functions to the preconditioned signal pattern until a predetermined degree of convergence is achieved so as to identifying events of interest within the enhanced signal pattern.

58. A method for analyzing an arrayed signal pattern generated by an arrayed platform device to identify events of interest within the signal pattern comprising the steps of:
inputting expresser functions capable of extracting spectral invariants of events of interest associated with the array device;
preconditioning the arrayed signal pattern so as to convert the arrayed signal pattern to a spectral domain; and
iteratively and selectively convolving the preconditioned signal pattern and the expresser functions so as to interferometrically enhance portions of the preconditioned signal pattern associated with events of interest, if any, present within the preconditioned signal pattern until a predetermined degree of convergence is achieved so as to identifying events of interest within the enhanced signal pattern.

59. The method of claim 58 wherein the step of iteratively and selectively convolving the preconditioned signal pattern and the expresser functions is performed by repeatedly convolving a single fixed expressor function to the preconditioned signal pattern using reverberant convergence until the predetermined degree of convergence is achieved.

60. The method of claim 58 wherein the step of iteratively and selectively convolving the preconditioned signal pattern and the expressor functions is performed by selectively convolving a set of different fixed expressor functions to the preconditioned signal pattern using reverberant convergence until the predetermined degree of convergence is achieved.

61. The method of claim 58 wherein the step of iteratively and selectively convolving the preconditioned signal pattern and the expressor functions is performed by selectively modifying and then convolving a dynamic expresser function to the preconditioned signal pattern using reverberant convergence until the predetermined degree of convergence is achieved.

62. A system for analyzing an arrayed signal pattern generated by an arrayed platform device to identify events of interest within the signal pattern comprising:
means for inputting expresser functions capable of extracting spectral invariants of events of interest associated with the array device;
means for preconditioning the arrayed signal pattern so as to convert the arrayed signal pattern to a spectral domain; and
means for iteratively and selectively convolving the preconditioned signal pattern and the expresser functions so as to interferometrically enhance portions of the preconditioned signal pattern associated with events of interest, if any, present within the preconditioned signal pattern until a predetermined degree of convergence is achieved so as to identifying events of interest within the enhanced signal pattern.

63. A computer code product for actively analyzing a signal pattern representative of arrayed data to identify events of interest therein, comprising:
computer code that inputs a signal pattern representative of arrayed data;
computer code that generates resonance patterns based on interference between synthetic noise and the signal pattern; and
computer code that detects resonances within the resonance patterns associated with events of interest.

64. A computer code product for analyzing an arrayed signal pattern generated by an arrayed platform device to identify events of interest within the signal pattern, comprising:
computer code that preconditions the arrayed signal pattern based on input expresser functions so as to convert the arrayed signal pattern to a spectral domain in which spectral harmonics parameterize events of interest to a predetermined dynamical system;
computer code that convolves the preconditioned signal pattern and the expressor functions so as to interferometrically enhance portions of the preconditioned signal pattern associated with events of interest, if any, present within the preconditioned signal pattern; and
computer code that identifies the occurrence of events of interest within the enhanced signal pattern.
